(19) United States
(12) Patent Application Publication
(10) Pub. No.: US 2025/0057542 A1
(43) Pub. Date: Feb. 20, 2025

(54) COLLAR LOCK AND METHOD FOR LOCKING A SHAFT WITHIN A HOUSING

(71) Applicant: MEDTRONIC PS MEDICAL, INC., Fort Worth, TX (US)

(72) Inventors: Hubert B. Beamon, Haltom City, TX (US); Michael Vu, Grand Prairie, TX (US); Jennifer M. Cao, Mckinney, TX (US); Aayush Malla, Fort Worth, TX (US); Saideep Nakka, Northlake, TX (US)

(21) Appl. No.: 18/761,368

(22) Filed: Jul. 2, 2024

Related U.S. Application Data
(60) Provisional application No. 63/533,186, filed on Aug. 17, 2023.

(51) Int. Cl. A61B 17/16; A61B 17/00

(52) U.S. Cl. CPC A61B 17/1613; A61B 2017/00477

(57) ABSTRACT

A surgical device includes a housing configured to receive a tool having a cylinder configured to support a drive shaft. A collar assembly is disposed atop the housing and is configured to engage a cylinder head of the cylinder. The collar assembly includes: a collar having a step ring separating an inner peripheral surface and a spring assembly seat, the inner peripheral surface dimensioned to engage the housing and the spring assembly seat configured to receive a spring assembly therein. One or more engagement spheres are configured to seat within a ball pocket defined through the housing. The sphere(s) is configured on one end to abut the cylinder head upon introduction into the housing and on the opposite end to seat within a groove defined within the collar. The sphere(s) translates within the groove between first and second pockets upon collar rotation to secure the cylinder within the housing.

### CROSS-REFERENCE TO RELATED APPLICATION

This application claims the benefit of and priority to U.S. Provisional Patent Application No. 63/533,186 filed Aug. 17, 2023, the entire disclosure of which is incorporated by reference herein.

### FIELD

The present disclosure is generally directed to devices and systems for cutting and treating bone and hard tissue and biomaterials. More particularly, the devices and systems of the present disclosure may be particularly suitable for orthopedical applications and other surgical procedures requiring the attachment and locking of shaft-driven surgical tools onto motor driving hand-held housings for enabling bone removal.

### BACKGROUND

Devices and systems in accordance with the present disclosure may be suitable for a variety of procedures including orthopedical surgical procedures, spinal procedures, cranial procedures, and other procedures requiring bone or hard tissue removal. During a given procedure, a motor is used to power a drill disposed at a distal end of the surgical tool. Typically, the tool is rotated at a very high RPM which allows the drill dissecting tip of the tool to plunge into the bone or, in some instances, cut or shave the bone using one or more flutes on the drill dissecting tip. In many instances it is important to align and lock the shaft of the rotating tool within a housing or bore during assembly to mechanically engage one or more internal components disposed within the housing, e.g., a drive shaft. It is important that that the user be able to quickly and confidently engage and lock the tool as needed and, equally important that the user be able to quickly uncouple the tool as needed.

### SUMMARY

Provided in accordance with the present disclosure is a surgical device which includes an outer housing configured to receive a surgical tool within a cavity defined within the outer housing, the surgical tool including a cylinder configured to support a drive shaft for rotation therein. The surgical device also includes a collar assembly disposed atop the outer housing and configured to engage a cylinder head of the cylinder. The collar assembly includes: a collar having a step ring separating an inner peripheral surface and a spring assembly seat, the inner peripheral surface dimensioned to engage a first stepped surface atop the outer housing and the spring assembly seat configured to receive a spring assembly therein; and one or more engagement spheres configured to operably seat within a corresponding ball pocket defined through the outer housing into the cavity, the engagement sphere(s) configured on one end to abut the cylinder head of the cylinder upon introduction of the cylinder head into the housing and on the opposite end to seat within a complementary one or more grooves defined within the inner peripheral surface of the collar, the engagement sphere(s) configured to guide the collar along the groove such that the engagement sphere transitions between a first groove pocket and a second groove pocket upon rotation of the collar to secure the cylinder within the housing.

In aspects according to the present disclosure, the one or more grooves vary in depth between the first groove pocket and the second groove pocket such that more force is imparted by the engagement sphere(s) on the cylinder upon rotation of the collar.

In aspects according to the present disclosure, the first groove pocket and the second groove pocket vary in depth. In other aspects according to the present disclosure, an angle of depth between the first groove pocket and the second groove pocket ranges from about 30 degrees to about 100 degrees.

In aspects according to the present disclosure, the spring assembly includes a spring disposed configured to provide a bias between the cylinder and the housing.

In aspects according to the present disclosure, the spring assembly includes a locking ring configured to retain the collar assembly atop the outer housing.

In aspects according to the present disclosure, the cylinder head includes one or more recesses defined in the outer surface thereof, the one or more recesses configured to complement the first groove pocket on the opposite end the engagement sphere(s) to at least partially retain the engagement sphere(s) between the recess and first groove pocket when the cylinder head fully engages within the outer housing.

In aspects according to the present disclosure, the surgical tool includes an alignment tab extending therefrom, the alignment tab configured to operably engage a notch defined in the outer housing when the cylinder head fully engages within the outer housing, the alignment tab and notch configured to prevent rotation of the surgical tool during rotation of the collar.

In aspects according to the present disclosure, a center axis of each groove is offset relative to an axis of rotation of the collar.

In aspects according to the present disclosure, an angle of rotation of the collar within each one or more grooves to transition the corresponding one or more engagement spheres between the first groove pocket and the second grove pocket ranges from about 15 degrees to about 300 degrees.

### BRIEF DESCRIPTION OF THE DRAWINGS

In the drawings, where like numerals refer to like components throughout several views:

FIG. 1 is a perspective view of a collar locking assembly shown in a fully rotated locked position with a shaft cylinder of a surgical tool engaged and locked within an outer tool housing;

FIG. 2 is an exploded view of the collar locking assembly of FIG. 1;

FIG. 3A is a side, cross-sectional view of the collar locking assembly, outer housing and surgical tool of FIG. 1 shown during initial engagement of the shaft cylinder within the outer housing;

FIG. 3B is an enlarged view of the area of detail of FIG. 3A showing one of a series of engagement spheres disposed within an outer pocket defined within the outer housing;

FIG. 4A is a side, cross-sectional view of the shaft cylinder during initial movement F1 into the outer housing against the bias of a spring of the collar locking assembly forcing the engagement spheres into first pockets defined within grooves of the collar locking assembly;

FIG. 4B is front, cross-sectional view of a collar of the collar locking assembly showing the engagement spheres being forced into the first pockets defined within the groove of the collar locking assembly shown in FIG. 4A;

FIG. 5A is a side, cross-sectional view of the shaft cylinder during continued movement F2 into the outer housing against the bias of the spring of the collar locking assembly forcing the engagement spheres into the first pockets defined within grooves of the collar locking assembly;

FIG. 5B is front, cross-sectional view of the collar of the collar locking assembly showing the engagement spheres being forced into first pockets defined within the grooves of the collar locking assembly shown in FIG. 5A;

FIG. 6 is a side, cross-sectional view of the shaft cylinder during continued movement F3 into the outer housing against the bias of the spring of the collar locking assembly forcing the engagement spheres into the first pockets defined within grooves of the collar locking assembly to a soft locking position and where the shaft cylinder bottoms out against the internal face of the outer housing and a locking tab in the shaft cylinder mechanically seats within a notch defined within the outer housing;

FIGS. 7A-7C are various views of the collar of the collar locking assembly upon initial rotation from the soft locking position to the fully locked position;

FIGS. 8A-8B are various views of the collar of the collar locking assembly in a fully rotated and locked position RI, with the engagement spheres seated within the second pockets; and

FIGS. 9A and 9B are various views of the collar of the collar locking assembly showing the details of the grooves and an example of a rotation angle for going from a soft locked position to a locked position with the collar locking assembly.

### DETAILED DESCRIPTION

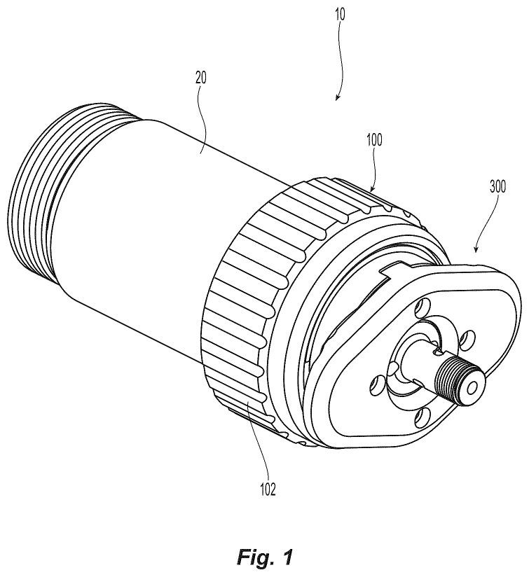

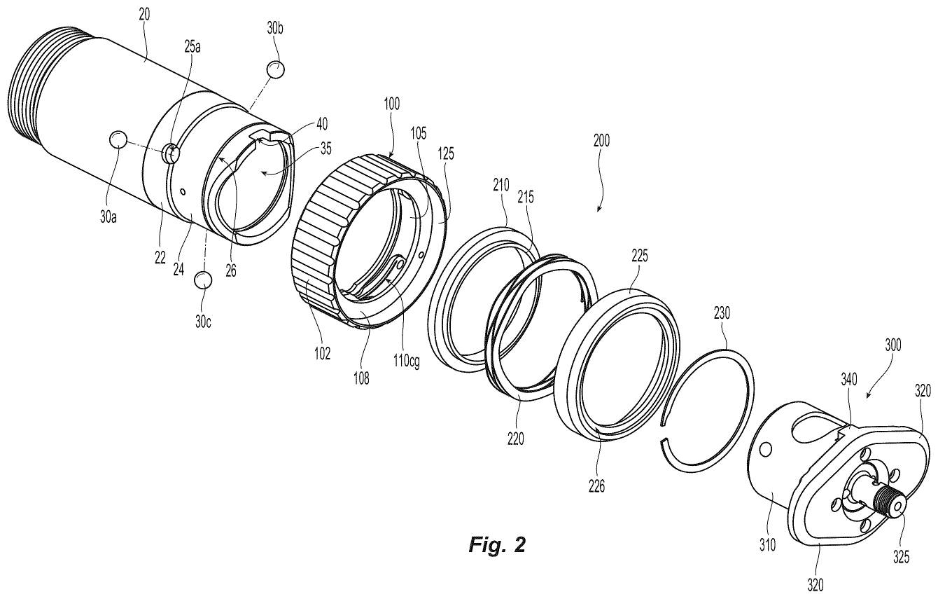

FIGS. 1-2 illustrate one embodiment of an assembled surgical device 10 configured for use in orthopedic and cranial surgical procedures for dissecting, cutting, shaving, and otherwise removing bone and hard tissue. Device 10 includes an outer housing 20, a collar assembly 100, and a surgical tool 300. Outer housing 20 is configured to be handled by a surgeon (or robot-assisted) and typically includes a motor (not shown) operably associated therewith. The surgical tool 300 aligns and operably engages the outer housing 20 and includes, inter alia, a drive shaft 325, cylinder head 310, and an outer flange 320. The collar assembly 100 locks the surgical tool 300 atop the outer housing 20.

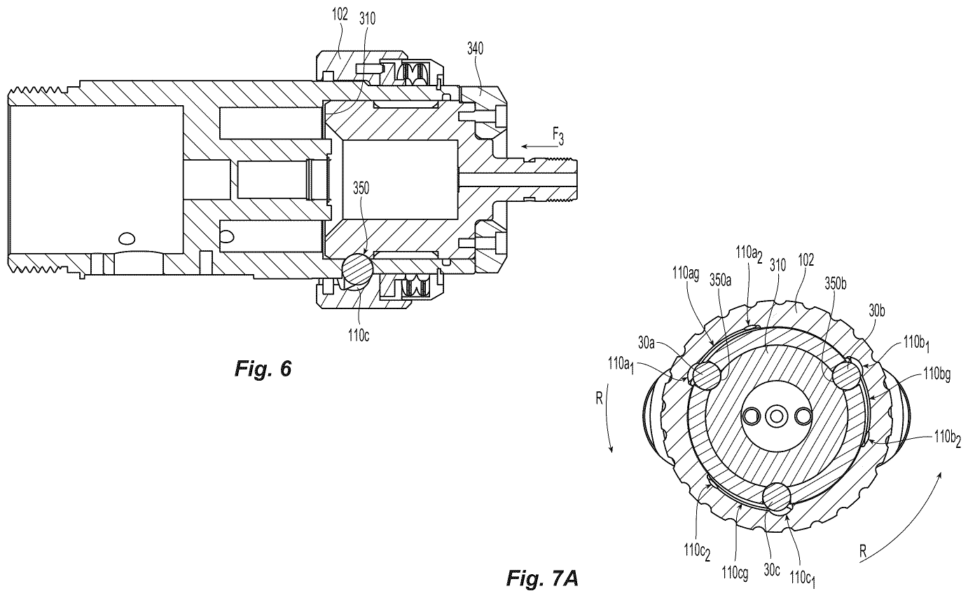

Drive shaft 325 is configured to align and operably engage a corresponding drive shaft (not shown) or other drive mechanism disposed within the outer housing 20. Cylinder head 310 is configured to be operably received within an internal bore 35 defined within the outer housing 20 and an alignment tab 340 disposed on the flange 320 is configured to mechanically engage a corresponding notch 40 defined within the outer housing 20 when the cylinder 310 is fully engaged therein as explained in more detail below with respect to FIG. 6.

Outer housing 20 is substantially cylindrical and includes first and second stepped surfaces 22 and 24 disposed thereabout configured to support a collar 102 of the collar assembly 100 and a spring assembly 200 disposed within the collar assembly 100. The first stepped surface 22 includes a series of openings defined therein (only opening 25a is shown in FIG. 2) configured to house an engagement sphere, e.g., sphere 30a, therein, the purposes of which is explained in detail below. The second stepped surface 24 is configured to abut a stop disposed along the inner peripheral surface of collar 102 on a proximal side thereof and abut a spring post 210 of the spring assembly 200 on the other side thereof (which also corresponds with the engagements spheres 30a-30c engaging respective grooves 110ag-110cg as detailed below).

Spring assembly 200 includes the spring post 210 which has an inner mounting flange 215 that is configured to support a compression spring 220 thereabout. A spring housing 225 snaps (or otherwise mechanically engages) spring post 210 encompassing the spring 220 therein. The spring housing 225 is configured to seat within an inner peripheral cavity 125 defined within the collar 102. Upon assembly, the spheres 30a-30c are seated within the corresponding openings, e.g., opening 25a shown, and the collar 102 with spring assembly 200 are slid atop outer housing 20 until stop 108 abuts the proximal end of step 24. Alternatively as mentioned above, engagements spheres 30a-30c may engage respective grooves 110ag-110cg to seat the collar 125. A locking ring 230 engages a distal step 26 of the outer housing 20 to lock the collar assembly 100 and spring assembly 200 atop the outer housing 20. The locking ring 230 is configured to seat within a ring cavity 226 defined about the distal end of the spring housing 225.

Collar assembly 100 includes the collar 102 and defines an inner peripheral surface 105 on the proximal side of the stop 108 that includes a series of grooves 110ag, 110bg and 110cg defined therein each including first and second respective groove pockets defined at the terminal ends thereof, e.g., groove 110ag including first groove pocket 110a1 and second groove pocket 110a2, groove 110bg including first groove pocket 110b1 and second groove pocket 110b2, and groove 110cg including first groove pocket 110c1 and second groove pocket 110c2. The first groove pocket, e.g., first pocket 110c1, associated with each groove, e.g., groove 110cg, is generally deeper than the second groove pocket, e.g., pocket 110c2, and is configured for a so-called “soft locked” position of the collar assembly 100 as explained in more detail below. Hereinafter groove pockets are simply referred to as “pockets”.

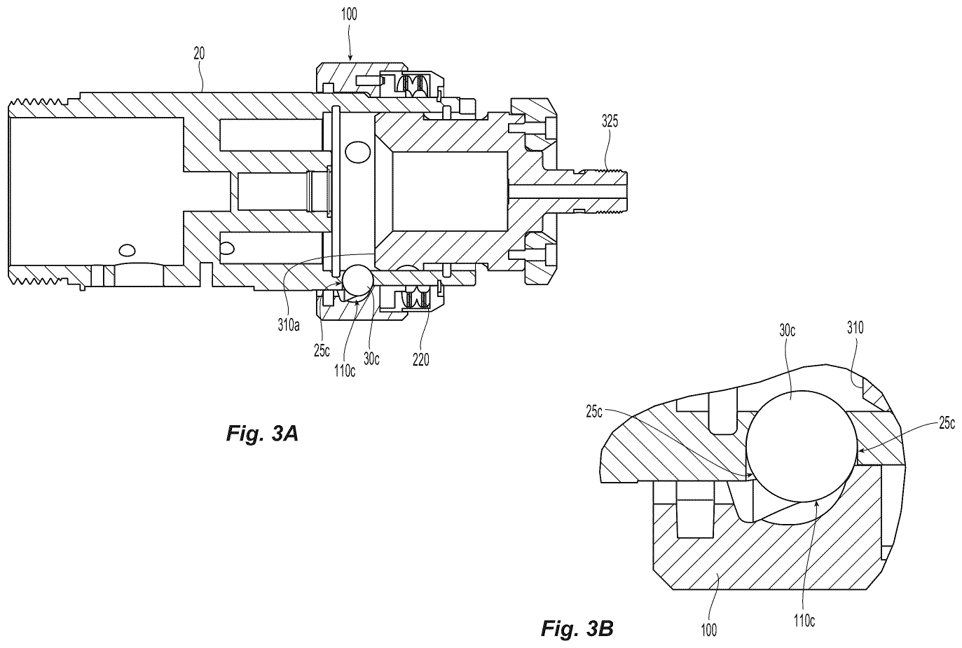

Turning now to FIGS. 3A and 3B which show the cylinder head 310 upon initial engagement within the outer housing 20 (prior to engagement with the engagement spheres 30a-30c (hereinafter “spheres 30a-30c”)), the spheres 30a-30c remain lodged within their individual openings, e.g., openings 25a and 25c shown, in the outer housing 20 with minimal contact with the collar 102 in this position. Moreover, very little force “F” is exerted on the spring 220 (if any). As shown in FIG. 3B, a distal face 310a of the cylinder head 310 remains spaced from the spheres, e.g., sphere 30c.

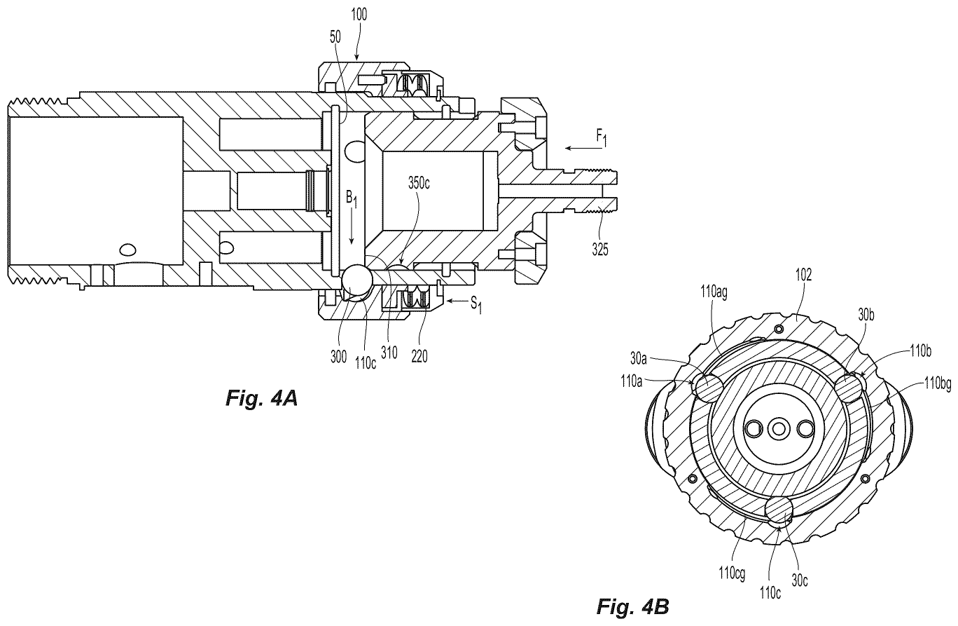

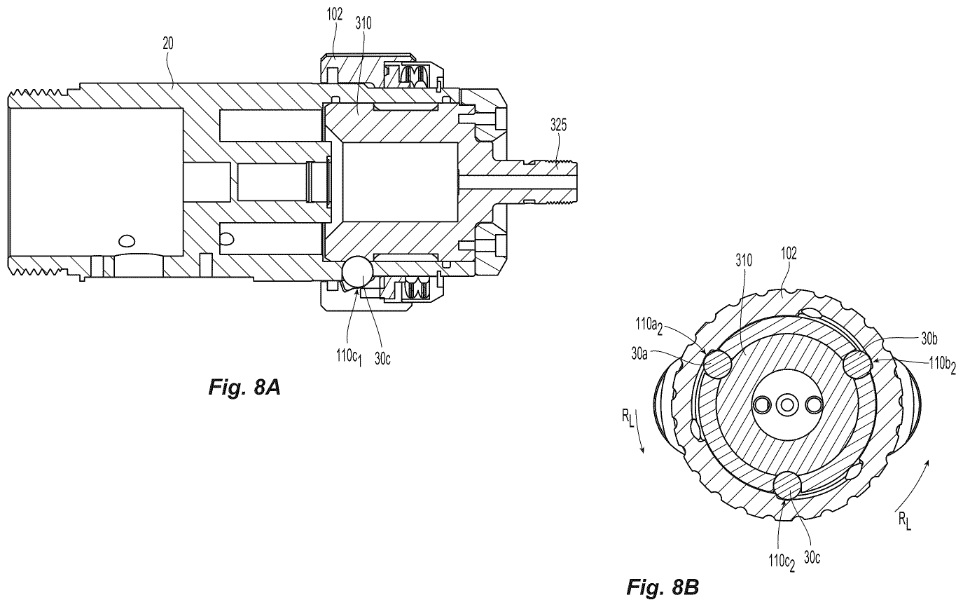

The tool 300 is engaged and locked within the outer housing 20 in two separate stages or in two locking positions, namely, a so-called “soft locked” position which is, ultimately, shown in FIG. 6 and so-called “fully locked” position as shown in FIGS. 8A and 8B. The locking positions are sequential. To initially engage the tool 300 within the outer housing 20 in soft locked engagement, the user exerts a force F₁ on the tool 300 against spring 220 in the direction S₁ forcing the cylinder head 310 into the internal bore 35 within the outer housing 20. The distal end 310a of the cylinder head 310 contacts the spheres 30a-30c forcing the spheres 30a-30c into respective first pockets 110a1-110c1 defined within respective grooves 110ag-110cg of collar 102 in the direction B₁ (FIGS. 4A and 4B). The cylinder head 310 also defines a series of soft lock recesses 350a-350c which are configured to align and seat each sphere 30a-30c when each sphere 30a-30c reaches the soft locked position as shown in FIG. 6.

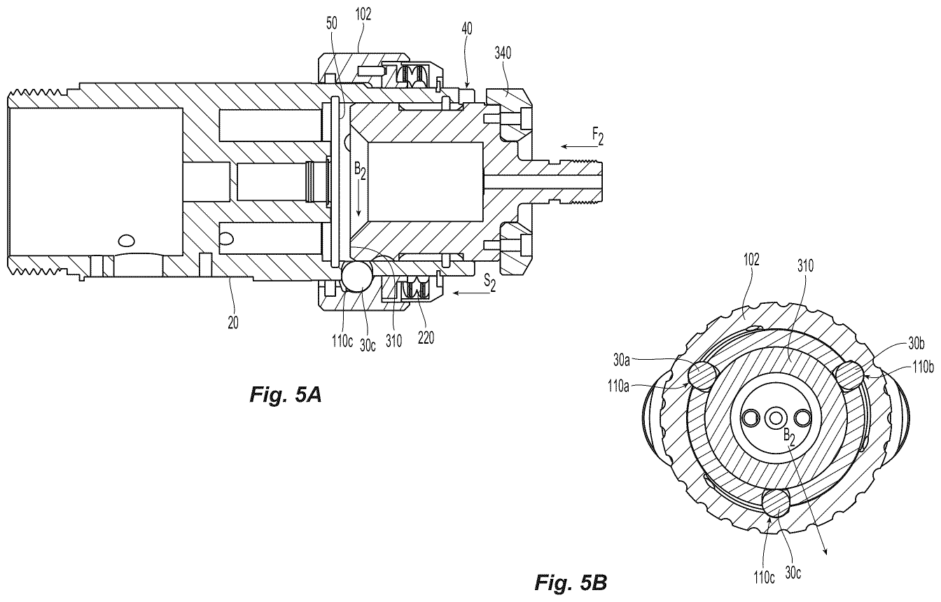

Continued movement of the tool 300 towards the soft locked position is shown in FIGS. 5A-5B wherein the tool 300 is forced further into bore 35 as the user exerts additional force F₂ (or continues to exert force F₁) on tool 300 against spring 220 in the direction S₂ forcing the cylinder head 310 further into the internal bore 35 within the housing 20. The cylinder 310 further forces the spheres 30a-30c into respective first pockets 110a1-110c1 defined within respective grooves 110ag-110cg of collar 102 in the direction B₂ (FIGS. 5A and 5B). Alignment tab 340 of cylinder head 310 also moves closer to engagement with notch 40 of outer housing 20.

As mentioned above, FIG. 6 shows the soft locked position of the tool 300 within the outer housing 20 wherein the cylinder head 310 is fully inserted within the internal bore 35 so that the distal end 310a abuts against faceplate 50 (FIGS. 4A and 5A) of internal bore 35. Other designs are contemplated including one or more flanges 320 on the cylinder. More particularly, the user exerts a force F₃ on the tool 300 against spring 220 in the direction S₃ (or continues to exert forces F₁ or F2) forcing the cylinder head 310 into the internal bore 35 against the spring 220 within the outer housing 20. The cylinder head 310 forces the spheres 30a-30c into respective first pockets 110a1-110c1 and grooves 110ag-110cg until the point that recesses 350a-350c seat the spheres 30a-30c therein when in the soft locked position. The notch 40 in the outer housing 20 receives the alignment member 340 of the cylinder head 310 which is designed to restrict the movement during rotation of the collar 102 as explained in more detail below. When disposed in the soft locked position, the spheres 30a-30c retain the tool 300 within the outer housing 20 with enough force to allow manipulation and handling thereof.

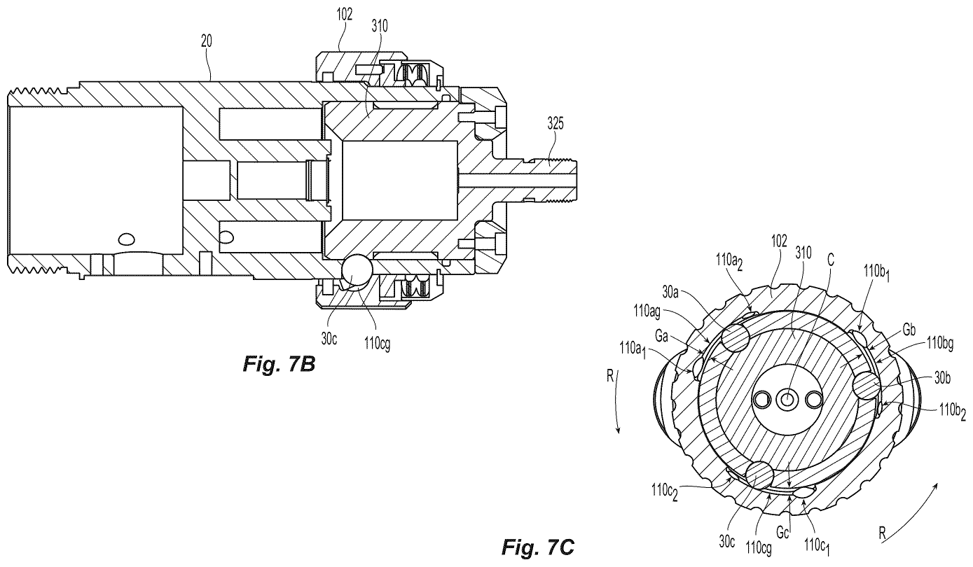

Turning now to FIGS. 7A-7C, the user rotates the collar 102 to move the tool 300 between the soft locked position and the fully locked position. Initial rotational movement in the direction “R” of the collar 102 overcomes the forces required to dislodge the collar 102 and sphere 30a-30c engagement from within first pockets 110a1-110c1. Each sphere 30a-30c guides the collar 102 along a corresponding groove 110ag-110cg as each sphere transitions towards a respective second pocket 110a2-110c2. The spheres 30a-30c remain within the recesses 350a-350c of cylinder 310 as the collar 102 rotates. Grooves 110ag-110cg are configured to vary in depth relative to the center axis “C” of the cylinder 310 (e.g., radius of the outer peripheral edge of each groove 110ag-110cg varies) such that a respective gap Gₐ, G_{b}, G_{c} defined therebetween decreases towards the second pocket 110a2-110c2. As a result thereof, continued rotational movement of the collar 102 in the direction R enables the spheres 30a-30c to provide more inward locking force between the tool 300 and the outer housing 20.

FIGS. 8A and 8B show the tool 300 in a locked position with the collar 102 fully rotated to the R_{L} position and the spheres 30a-30c seated within the second pockets 110a2-110c2. The tool 300 is now locked and ready for use. Turning the collar 102 in the opposite direction of R will move the collar 102 along the spheres 30a-30c from the fully locked position to the soft locked position.

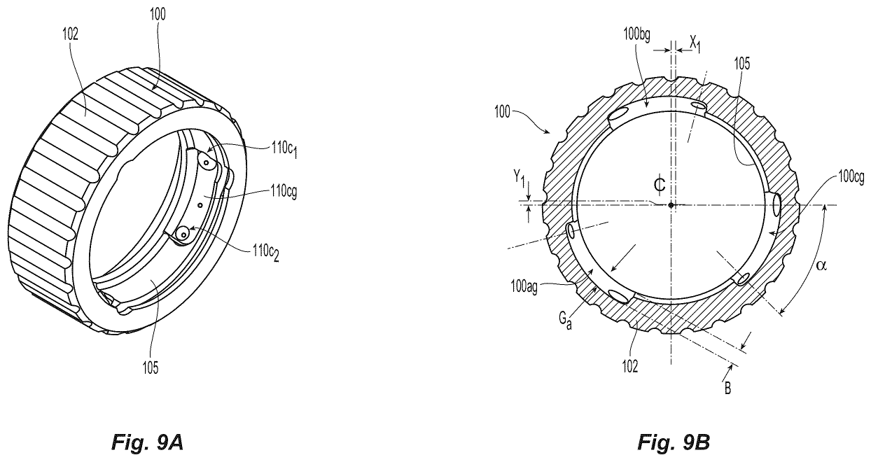

FIGS. 9A and 9B show the collar assembly 100 including the internal components associated therewith including the first and second pockets, e.g., pockets 110c1 and 110c2, groove 110cg and the inner peripheral surface 105. FIG. 9B shows the center axis “C” of the cylinder (which translates through to the collar 102) and also the envisioned radial offsets of the grooves 110ag-110cg along both the X and Y axes relative thereto, e.g., an offset distance X1 along the X axis and an offset distance Y1 along the Y axis. The collar 102 includes a rotation angle α from the soft locked position to the locked position. Angle α may vary from about 15 degrees to about 300 degrees. Alternatively, the grooves 110ag-110cg may be twisted to induce a higher spring force in the fully locked position compared to the soft locked position which would eliminate the need for an X or Y offset.

The gap G, e.g., gap Gₐ, may vary an angle β from about 30 degrees to about 100 degrees between the pockets 110c1 and 110c2. The pockets, e.g., pockets 110c1 and 110c2, may be varied in depth to facilitate locking and unlocking in both the soft locked position and the fully locked position. The fully locked pocket, e.g., pocket 110c2, may be shallower than the soft locked pocket, e.g., pocket 110c1, due to the overall locking pressure imparted by the spheres 30-30c against the cylinder head 310 associated with the change in gap G during rotation.

While several aspects of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular configurations. Those skilled in the art will envision other modifications within the scope and spirit of the claims appended hereto.

What is claimed is:

1. A surgical device, comprising:
an outer housing configured to receive a surgical tool within a cavity defined within the outer housing, the surgical tool including a cylinder configured to support a drive shaft for rotation therein; and
a collar assembly disposed atop the outer housing and configured to engage a cylinder head of the cylinder, the collar assembly including:
a collar including a step ring separating an inner peripheral surface and a spring assembly seat, the inner peripheral surface dimensioned to engage a first stepped surface atop the outer housing and the spring assembly seat configured to receive a spring assembly therein; and
at least one engagement sphere configured to operably seat within a corresponding ball pocket defined through the outer housing into the cavity, the at least one engagement sphere configured on one end to abut the cylinder head of the cylinder upon introduction of the cylinder head into the outer housing and on the opposite end to seat within a complementary at least one groove defined within the inner peripheral surface of the collar, the at least one engagement sphere configured to guide the collar along the at least one groove such that the at least one engagement sphere transitions between a first groove pocket and a second groove pocket upon rotation of the collar to secure the cylinder within the housing.

2. The surgical device according to claim 1, wherein the at least one groove varies in depth between the first groove pocket and the second groove pocket such that more force is imparted by the at least one engagement sphere on the cylinder upon rotation of the collar.

3. The surgical device according to claim 1, wherein the first groove pocket and the second groove pocket vary in depth.

4. The surgical device according to claim 1, wherein an angle of depth between the first groove pocket and the second grove pocket ranges from about 30 to about 100.

5. The surgical device according to claim 1, wherein the spring assembly includes a spring configured to provide a bias between the cylinder and the outer housing.

6. The surgical device according to claim 1, wherein the spring assembly includes a locking ring configured to retain the collar assembly atop the outer housing.

7. The surgical device according to claim 1, wherein the cylinder head includes at least one recess defined in the outer surface thereof, the at least one recess configured to complement the first groove pocket on the opposite end of the at least one engagement sphere to at least partially retain the at least one engagement sphere between the at least one recess and the first groove pocket when the cylinder head fully engages within the outer housing.

8. The surgical device according to claim 1, wherein the surgical tool includes an alignment tab extending therefrom, the alignment tab configured to operably engage a notch defined in the outer housing when the cylinder head fully engages within the outer housing, the alignment tab and notch configured to prevent rotation of the surgical tool during rotation of the collar.

9. The surgical device according to claim 1, wherein a center axis of each groove is offset relative to an axis of rotation of the collar.

10. The surgical device according to claim 1, wherein an angle of rotation of the collar within each at least one groove to transition the at least one engagement sphere between the first groove pocket and the second grove pocket ranges from about 15 degrees to about 300 degrees.
